(19) United States
(12) Patent Application Publication
(10) Pub. No.: US 2025/0018173 A1
(43) Pub. Date: Jan. 16, 2025

(54) Sensory Control System

(71) Applicant: Steve O'Neal, Oak Creek, CO (US)

(72) Inventor: Steve O'Neal, Oak Creek, CO (US)

(21) Appl. No.: 18/766,408

(22) Filed: Jul. 8, 2024

Related U.S. Application Data
(60) Provisional application No. 63/525,940, filed on Jul. 10, 2023.

(51) Int. Cl. A61N 1/04; A61N 1/36

(52) U.S. Cl. CPC A61N 1/0456; A61N 1/0484; A61N 1/3603

(57) ABSTRACT

A sensory control system that encompasses an outer surface of a body part for applying low level electrical impulses to the outer surface to alter a physical sensation of the body part. The system including a garment constructed of a flexible material with an electrode disposed between the garment and the outer surface, wherein operationally the electrode comes into contact with an outer surface of a particular part of a human body of a user. Also included in the system is control circuitry that is in electrical communication with the electrode, wherein the control circuitry is operative to generate the electrical impulses with particular frequency, phase, pulse versus time amounts, voltage, and ampere levels at user safe low electrical power levels that are less than one-hundred milli-amps and no more than sixty volts.

### CROSS-REFERENCE TO RELATED APPLICATION

This application claims the benefit of U.S. provisional patent application Ser. No. 63/525,940 filed on Jul. 10, 2023, by Steve O'Neal of Oak Creek, Colorado, U.S.

### BACKGROUND OF INVENTION

The present invention relates generally to a system for sensory control using electrical signals. More specifically, the present invention relates to the field of various devices using Transcutaneous Electrical Nerve Stimulation (TENS) having the advantages of being non-invasive and portable, TENS is a low voltage electric current that is in contact with the user's skin usually through an adhesive patch electrode with low electrical power levels (typically using smaller batteries) that can be varied in frequency from 1 to 120 hertz, wherein the TENS is used to help block nerve cell transmission of pain sensation or raising the level of pain blocking endorphins, being in either case to reduce the sensation of pain. Users report lasting effect of TENS can be zero, i.e. with the electrical current being removed the pain returns and users also report up to a twenty-four hour period of sensed pain reduction from TENS after removal.

The main reported risk of TENS is from skin irritation from the adhesive patch electrode, other risks that are associated with anybody can have issues with electrical impulses such as pacemakers, pregnancy, cancer, epilepsy, blood clots (as blood circulation is increased by TENS), infected areas, near eyes or mouth, near genitals, or low sensation areas. Skin contact can also be with acupuncture needles. Unique characteristics of a TENS system will be in novel skin contact structures (various electrode types) along with various body-hugging structures (garments) to hold the TENS system electrodes in the desired position on or adjacent to the body.

In looking at the prior art in the TENS field, starting with U.S. Pat. No. 8,421,448 to Tran, that discloses a glove with finger sensors for gesture commands, thus not being TENS, but showing patent teaching on glove fingertip embedded electrodes.

Next, in United States Patent Application Publication Number US2011/0007035 to Shai, discloses small elastomeric wearables that contain various sensors and further in United States Patent Application Publication Number US2015/0140934 to Abdurrahman, discloses wrist electrical wearable that wirelessly control through motion signals.

Continuing in United States Patent Number U.S. Pat. No. 11,247,053 to Rajguru, discloses a TENS device for pain mitigation for foot, knee, ankle, and various other parts of the body, the novelty includes a stimulator electrode with signal control effectuated through sensory feedback, wherein the novelty is with the signal control and feedback system.

In addition, in United States Patent Number U.S. Pat. No. 11,235,142 to Gozani, discloses a TENS smart electrode in the form of a wrap strap for a body wearable with novelty in the electrode having a controller that senses proper skin contact, a timer/intensity curve of the signal, and an electrode replacement notice.

Further, in United States Patent Number U.S. Pat. No. 11,213,679 to Chen, discloses a portable TENS device that has Bluetooth to a smartphone app for control of battery state, signal level, signal strength limits, plus smartphone generated audio signals.

Moving onward, in United States Patent Number U.S. Pat. No. 11,997,998 to Simon, discloses a headache pain electrical impulse treatment system via an electrode to the neck of a user that wirelessly receives a pain treatment regimen from a mobile device.

Next, United States Patent Number U.S. Pat. No. 10,898,718 to Srivastava, discloses a sensor based pain management system having closed loop control for pain therapy, using two sensors and two circuits with a controller, this is to activate pain therapy based upon another user movement or event.

Further, in United States Patent Number U.S. Pat. No. 10,661,072 to Kern, discloses a TENS attachment with a handheld pistol grip attachment electrode that converts mechanical motion to an electrical current to deliver the TENS therapy, also in United States Patent Number U.S. Pat. No. 9,675,801 to Kong, discloses a TENS system with an electrode that is time measured to minimize skin irritation.

Continuing, in United States Patent Number U.S. Pat. No. 8,868,216 to Dunagan, discloses a TENS system with an electrode garment with built-in electrodes that have a non-conductive porous material between the electrode and the user's skin to minimize discomfort to the user's skin.

Also, in United States Patent Application Publication Number US2022/0007750 to Ho, discloses a garment structure for electrotherapy, having positional adjustment of the electrode within the garment and further with a second heating electrode also built in the garment to provide both electrotherapy and heating.

Next, in United States Patent Application Publication Number US2021/0205607 to Herb, discloses a TENS system using phase shifted waveforms with different voltages using an electrode array.

Further, in United States Patent Application Publication Number US2021/0121682 to Mbue, discloses a sock that delivers electrical impulses to the foot and ankle for the treatment of pain using TENS that is wirelessly controlled remotely.

There are numerous patents and applications in the TENS field, which includes not only pain management, but sensory control for bladder control, muscle movement, and feedback from the user either through the electrode or manually for controlling the electrical impulses that the user receives, plus variances in frequency, number of signals, electrode types, and the type of garment for the wearable portion that contains the electrodes being gloves, socks, bands, etc.

The novelty with TENS falls in the following areas; the electrical signal itself (varying frequencies, voltage, and amp levels, multiple signals, different phases, and so on for various therapeutic effects), electrode design and skin attachment (for administering TENS, efficacy, skin irritation issues, and so on), and the garment itself, (glove, sock, wrap, and so on for the position and holding of the electrode).

As noted in TENS literature, a significant issue is with the fact that the electrode needs direct contact with the skin to work properly—i.e. typically adhesively attached, so with gloves, sock, and for pets, would involve not so direct contact with the skin the TENS effectiveness could be compromised, however, this is off set by the benefit of the not so direct contact of the electrode with the user's skin which would reduce the skin irritation that can come with the adhesive from the electrode to the skin attachment,

### SUMMARY OF THE INVENTION

Broadly, the present invention is a sensory control system that is adapted to encompass an outer surface of a particular part of a human body of a user for applying electrical impulses to the outer surface to alter a physical sensation of the particular part of the human body for the user. The sensory control system including a semi planar garment is constructed of a flexible material that is adapted to attach to the outer surface of a particular part of the human body, wherein the semi planar garment is removably engageable to the outer surface of a particular part of the human body, the semi planar garment including a planar garment outer surface, a planar garment inner surface, a planar garment first end portion, and an opposing planar garment second end portion, wherein the semi planar garment substantially assumes a shape of a surrounding sidewall.

Further included in the sensory control system is an electrode disposed within the garment, wherein the electrode is affixed to the semi planar garment inner surface, wherein operationally the electrode comes into contact with an outer surface of a particular part of a human body of a user when the semi planar garment is attached to the outer surface of the particular part of the human body.

Also included in the sensory control system is control circuitry that is in electrical communication with the electrode, wherein the control circuitry is operative to generate the electrical impulses with particular frequency, phase, pulse versus time amounts, voltage, and ampere levels at user safe low electrical power levels that are less than one-hundred milli-amps and no more than sixty volts.

These and other objects of the present invention will become more readily appreciated and understood from a consideration of the following detailed description of the exemplary embodiments of the present invention when taken together with the accompanying drawings, in which;

### BRIEF DESCRIPTION OF DRAWINGS

FIG. 1 shows a thumb-oriented perspective view of the sensory control system specifically being the semi planar garment in the form of a glove for the human hand;

FIG. 2 shows a palm-oriented perspective view of the sensory control system specifically being the semi planar garment in the form of the glove for the human hand;

FIG. 3 shows section view cut 3-3 from FIGS. 1 and 2, wherein FIG. 3 shows the cross section of a single fingertip encompassed by the finger surrounding sidewall with the electrode shown disposed in contact with the fingertip and to the surrounding sidewall inner surface;

FIG. 4 shows section view cut 4-4 from FIGS. 1 and 2, wherein FIG. 4 shows the cross section of a single fingertip encompassed by the finger surrounding sidewall with the electrode shown disposed in contact with the fingertip and to the surrounding sidewall inner surface, wherein FIG. 4 is rotated ninety degrees to be perpendicular to the section cut view in FIG. 3 for reference;

FIG. 5 shows an upper perspective view of the sensory control system in the form of an orthotic arch support shoe insert sole that includes an electrode disposed on the foot side first surface;

FIG. 6 shows a use view of an upper perspective view of the sensory control system in the form of an orthotic arch support shoe insert sole with the user's foot in place;

FIG. 7 shows cross section cut 7-7 from FIG. 6, wherein FIG. 7 shows the electrode disposed between the foot outer surface and the first surface;

FIG. 8 shows cross section cut 8-8 from FIGS. 6 and 7, wherein FIG. 8 shows the electrode disposed between the foot outer surface and the first surface, wherein FIG. 8 is rotated ninety degrees to be perpendicular to the section cut view in FIG. 7 for reference;

FIG. 9 shows an upper perspective view of the sensory control system in the form of a pet grooming glove that includes an electrode disposed on the inside exterior surface;

FIG. 10 shows an approaching use upper perspective view of the sensory control system in the form of the pet grooming glove that includes the electrode disposed on the inside exterior surface, wherein the electrode has initial contact with the human head skin surface;

FIG. 11 shows a full contact use upper perspective view of the sensory control system in the form of the pet grooming glove that includes the electrode disposed on the inside exterior surface, wherein the electrode has full contact with the human head skin surface;

FIG. 12 shows cross section cut 12-12 from FIG. 11, wherein FIG. 12 shows a clearer view of the pet grooming glove that includes the electrode disposed on the inside exterior surface, wherein the electrode has full contact with the human head skin surface; and

FIG. 13 shows cross section cut 13-13 from FIG. 11, wherein FIG. 13 shows a clearer view of the pet grooming glove that includes the electrode disposed on the inside exterior surface, wherein the electrode has full contact with the human head skin surface, wherein FIG. 13 is rotated ninety degrees to be perpendicular to the section cut view in FIG. 12 for reference.

### DETAILED DESCRIPTION OF THE INVENTION

50 Sensory Control System
55 User
60 Human body
61 Human hand
62 Palm of the human hand 61
63 Back of the human hand 61
65 Particular portion of the human body 60
70 Outer surface of the human body 60
75 Particular part of the human body 60
76 Human foot
77 Arch skin surface of the human foot 76
78 Human head
79 Skin surface of the human head 78
80 Semi planar garment
85 Outer surface of the planar garment 80
90 Inner surface of the planar garment 80
95 First end portion of the planar garment 80
100 Second end portion of the planar garment 80
105 Surrounding sidewall of the planar garment 80
110 Electrode
111 Electrical impulses from the electrode 110
115 Affixed structure of the electrode 110 to the inner surface 90
120 Contact of the electrode 110 to the outer surface 70 of the particular part 75 of the human body 60
125 Attached position of the semi planar garment 80 to the outer surface 70 of the particular part 75 of the human body 60
130 Control circuitry
135 Electrical communication between the control circuitry 130 and the electrode 110 that can be in the form of wireless or wired
140 Human hand
145 Finger of the human hand 140
146 Fingertip of the human hand 140
150 Glove for the human hand 140
155 Plurality of adjacent surrounding sidewalls
160 Fourchettes of the glove 150
165 Thumb of the glove 150
170 Inner surface of each of the surrounding sidewalls 155
175 Affixed structure of the electrode 110 being disposed upon the inner surface 170
180 Contact of the electrode 110 upon the fingertip 146 of the human hand 140
185 Orthotic arch support shoe insert insole
190 Human foot side first surface of the orthotic arch support shoe insert insole 185
195 Shoe sole side second surface of the orthotic arch support shoe insert insole 185
200 Affixed structure of the electrode 110 being disposed upon the human foot side first surface 190 of the orthotic arch support shoe insert insole 185
205 Contact of the electrode 110 upon the arch skin surface 77 of the human foot
210 Pet grooming type of glove
215 Grooming glove surrounding sidewall
220 Encompass manner of the grooming glove surrounding sidewall 215 about the human hand palm 62 and human hand back 63
225 Exterior surface of the grooming glove surrounding sidewall 215
230 Inside exterior surface of the exterior surface 225
235 Outside exterior surface of the exterior surface 225
240 Adjacent nature of the inside exterior surface 230 to the palm 62 of the hand 61
245 Adjacent nature of the outside exterior surface 235 to the back 63 of the hand 61
250 Affixed structure of the electrode 110 being disposed upon the inside exterior surface 230 of the of the exterior surface 225
255 Contact of the electrode 110 upon the portion of the human head 78 skin surface 79

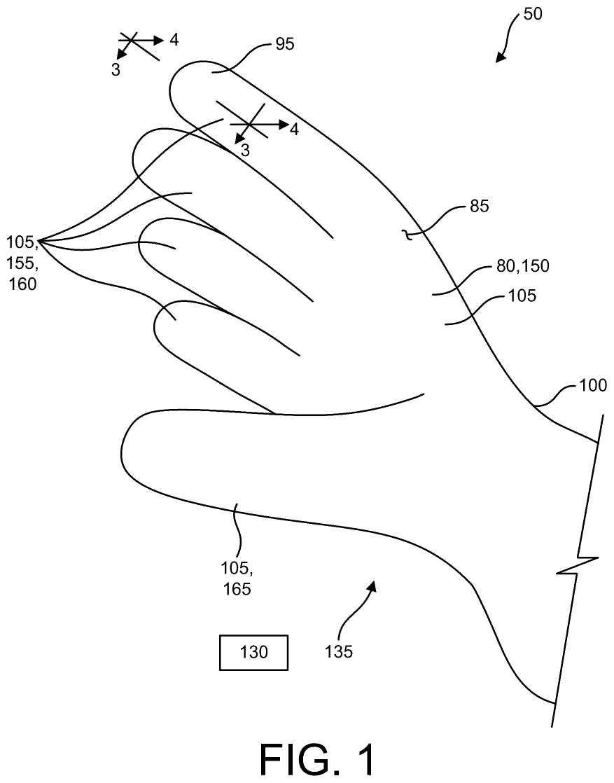

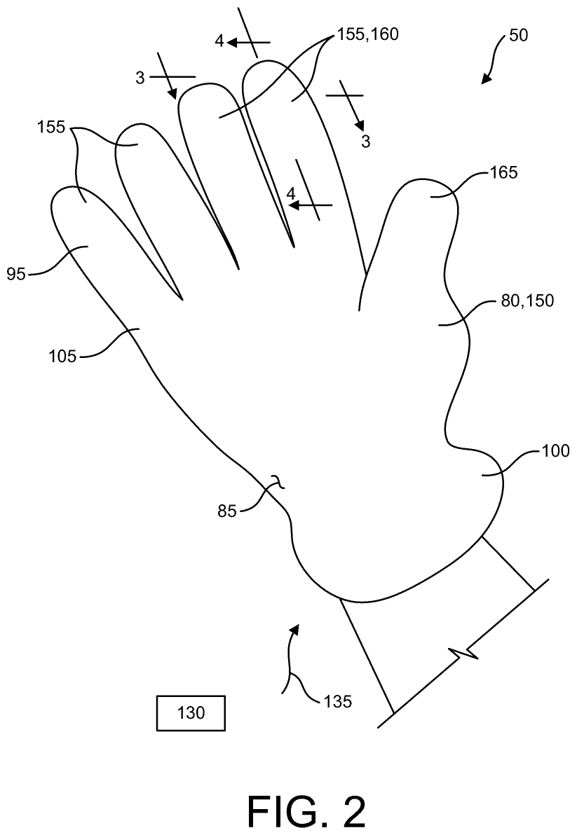

With initial reference to FIG. 1, shown is a thumb-oriented perspective view of the sensory control system 50 specifically being the semi planar garment 80 in the form of a glove for the human hand 150. Next, FIG. 2 shows a palm-oriented perspective view of the sensory control system 50 specifically being the semi planar garment 80 in the form of the glove for the human hand 150.

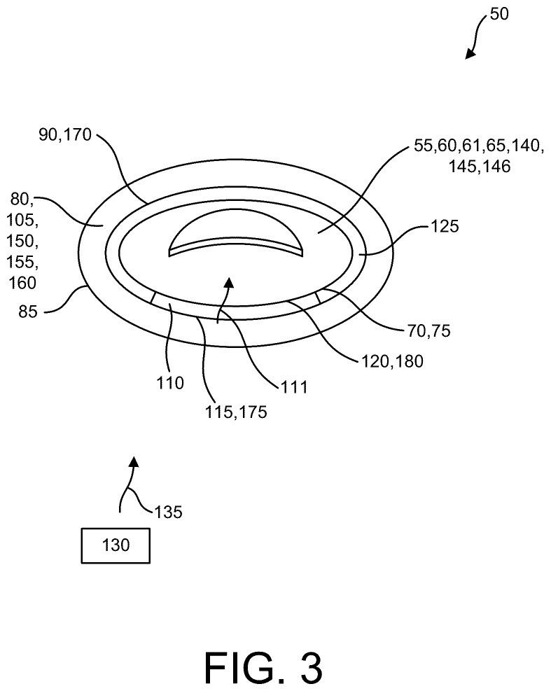

Further, FIG. 3 shows cross section view cut 3-3 from FIGS. 1 and 2, wherein FIG. 3 shows the cross section of a single fingertip 146 encompassed by the finger surrounding sidewall 155 with the electrode 110 shown disposed in contact 180 with the fingertip 146 and to the surrounding sidewall 155 inner surface 170.

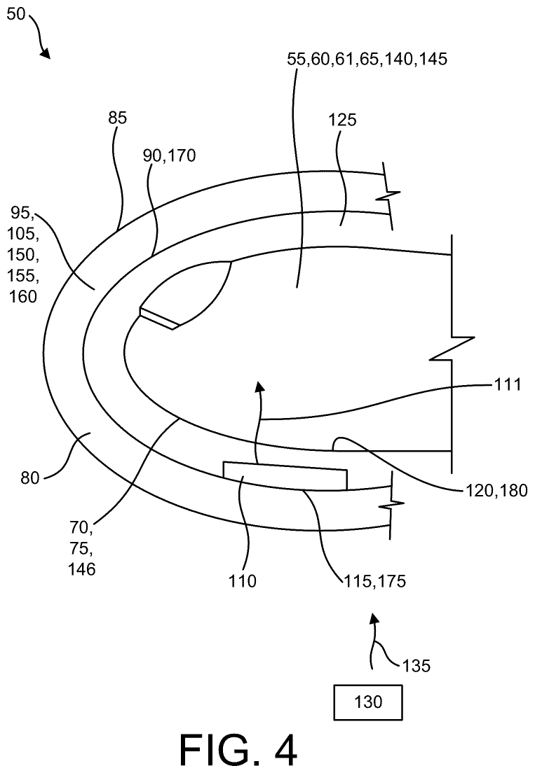

Continuing, FIG. 4 shows cross section view cut 4-4 from FIGS. 1 and 2, wherein FIG. 4 shows the cross section of a single fingertip 146 encompassed by the finger surrounding sidewall 155 with the electrode 110 shown disposed in contact 180 with the fingertip 146 and to the surrounding sidewall 155 inner surface 170, wherein FIG. 4 is rotated ninety degrees to be perpendicular to the cross section cut view in FIG. 3 for reference.

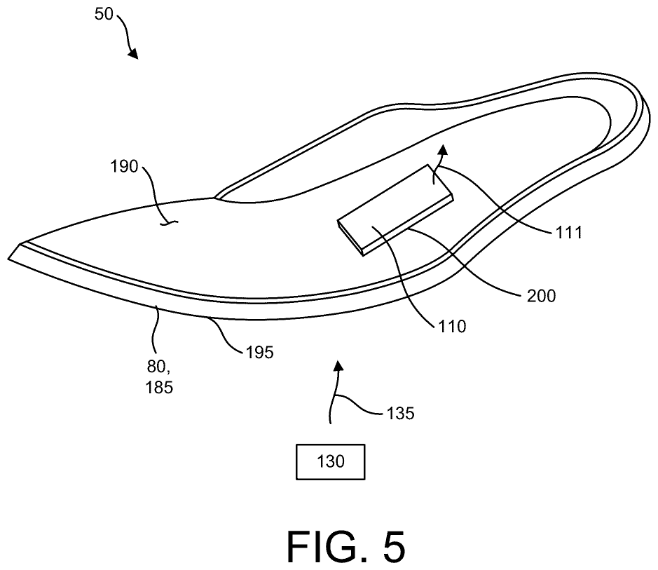

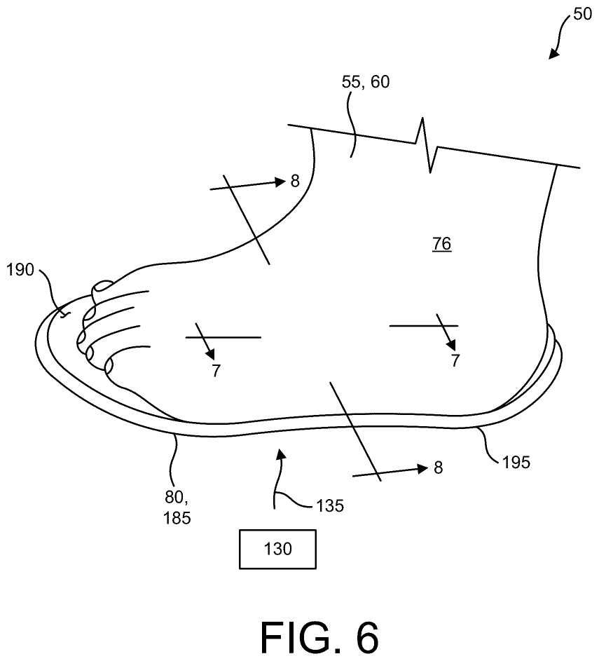

Next, FIG. 5 shows an upper perspective view of the sensory control system 50 in the form of an orthotic arch support shoe insert sole 185 that includes an electrode 110 disposed on the foot side first surface 190. Also, FIG. 6 shows a use view of an upper perspective view of the sensory control system 50 in the form of an orthotic arch support shoe insert sole 185 with the user's 55 foot 76 in place.

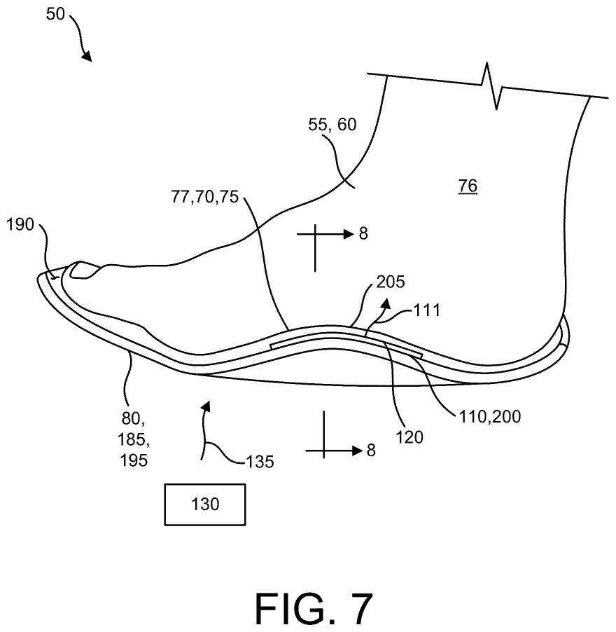

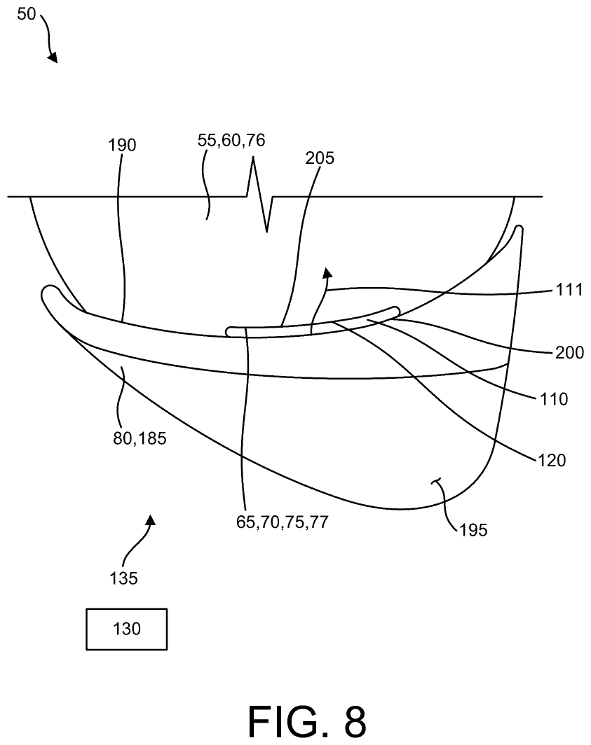

In addition, FIG. 7 shows section cut 7-7 from FIG. 6, wherein FIG. 7 shows the electrode 110 disposed between the foot outer surface 77 and the first surface 190. Next, FIG. 8 shows section cut 8-8 from FIGS. 6 and 7, wherein FIG. 8 shows the electrode 110 disposed between the foot outer surface 77 and the first surface 190, wherein FIG. 8 is rotated ninety degrees to be perpendicular to the section cut view in FIG. 7 for reference.

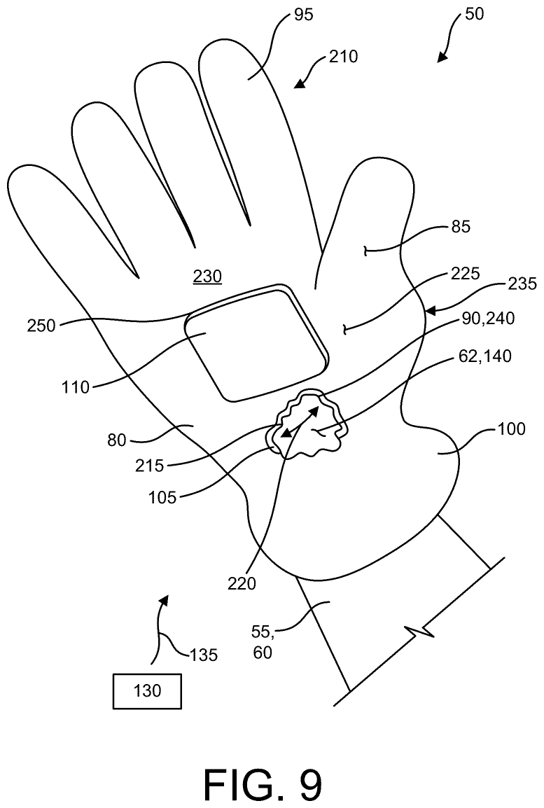

Continuing, FIG. 9 shows an upper perspective view of the sensory control system 50 in the form of a pet grooming glove 210 that includes an electrode 110 disposed on the inside exterior surface 230.

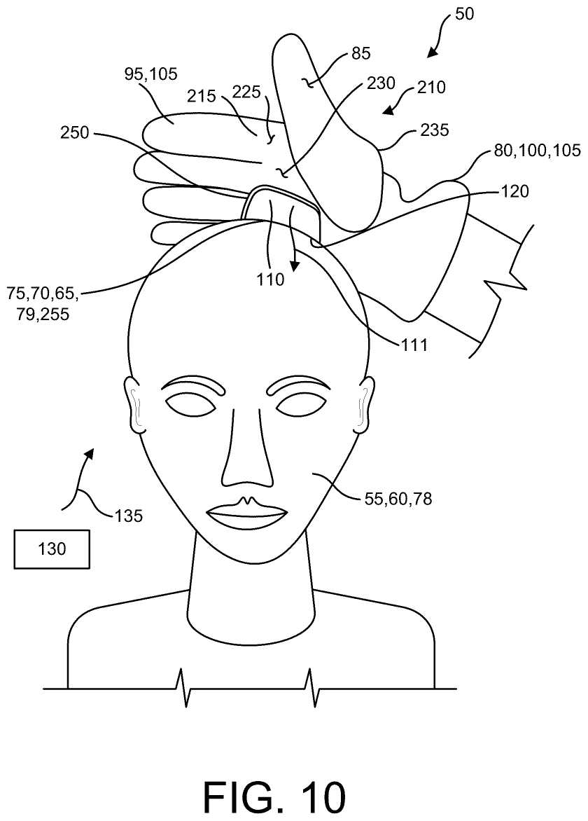

Further, FIG. 10 shows an approaching use upper perspective view of the sensory control system 50 in the form of the pet grooming glove 210 that includes the electrode 110 disposed on the inside exterior surface 230, wherein the electrode 110 has initial contact with the human head 78 skin surface 79.

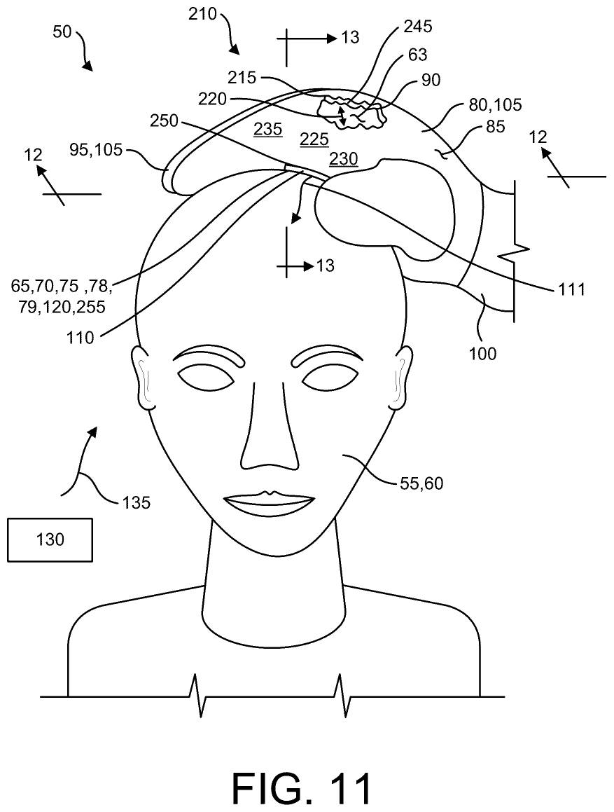

In addition, FIG. 11 shows a full contact use upper perspective view of the sensory control system 50 in the form of the pet grooming glove 210 that includes the electrode 110 disposed on the inside exterior surface 230, wherein the electrode 110 has full contact with the human head 78 skin surface 79.

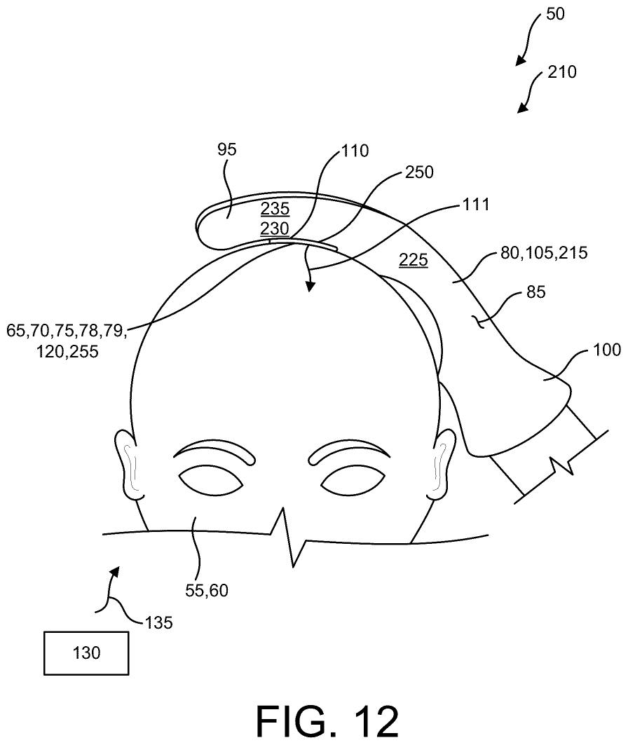

Continuing, FIG. 12 shows cross section cut 12-12 from FIG. 11, wherein FIG. 12 shows a clearer view of the pet grooming glove 210 that includes the electrode 110 disposed on the inside exterior surface 230, wherein the electrode 110 has full contact with the human head 78 skin surface 79.

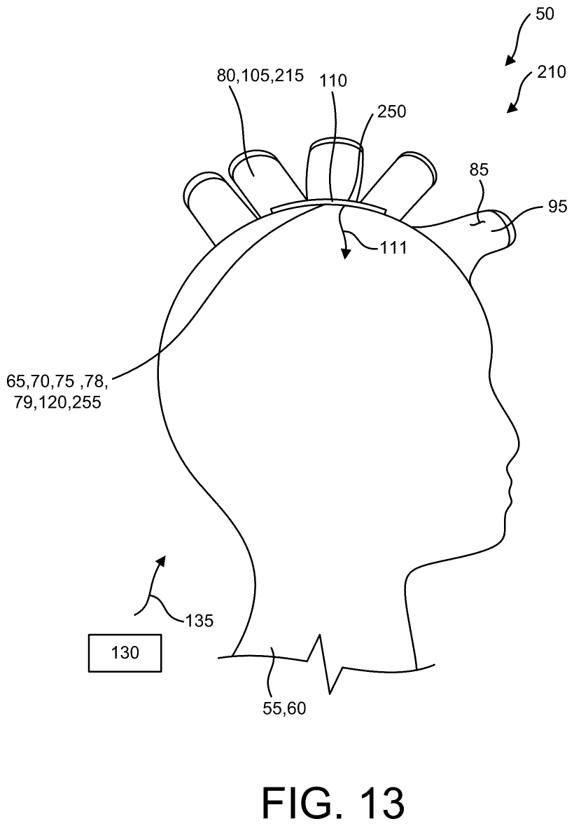

Next, FIG. 13 shows cross section cut 13-13 from FIG. 11, wherein FIG. 13 shows a clearer view of the pet grooming glove 210 that includes the electrode 110 disposed on the inside exterior surface 230, wherein the electrode 110 has full contact with the human head 78 skin surface 79, wherein FIG. 13 is rotated ninety degrees to be perpendicular to the section cut view in FIG. 12 for reference.

Broadly, in looking at FIGS. 1 to 13, the present invention is a sensory control system 50 that is adapted to encompass an outer surface 70 of a particular part 75 or portion 65 of the human body 60 of the user 55 for applying electrical impulses 111 to the outer surface 70 to functionally alter a physical sensation of the particular part 75 of the human body 60 for the user 55.

The sensory control system 50 including a semi planar garment 80 constructed of a flexible material that is adapted to attach to the outer surface 70 of the particular part 75 of the human body 60, wherein the semi planar garment 80 is removably engageable to the outer surface 70 of the particular part 75 of the human body 60, the semi planar garment 80 including a planar garment outer surface 85, a planar garment inner surface 90, a planar garment first end portion 95, and an opposing planar garment second end portion 100, wherein the semi planar garment 80 substantially assumes a shape of a surrounding sidewall 105.

Further included in the sensory control system 50 is an electrode 110 disposed within the garment 80, wherein the electrode 110 is affixed 115 to the semi planar garment inner surface 90, wherein operationally the electrode 110 comes into contact 120 with the outer surface 70 of the particular part 75 of the human body 60 of the user 55 when the semi planar garment 80 is attached 125 to the outer surface 70 of the particular part 75 of the human body 60.

Also included in the sensory control system 50 is control circuitry 130 that is in electrical communication 135 with the electrode 110, wherein the control circuitry 130 is operative to generate the electrical impulses 111 with particular frequency, phase, pulse versus time amounts, voltage, and ampere levels at user 55 safe low electrical power levels that are less than one-hundred milli-amps and no more than sixty volts.

As an option for the sensory control system 50, wherein the electrode 110 and the control circuitry 130 can be constructed of a transcutaneous electrical nerve stimulation (TENS) system.

A further option for the sensory control system 50, wherein the semi planar garment 80 can be constructed of a glove 150 for a human hand 61 that includes a plurality of adjacent surrounding sidewalls 155 forming fourchettes 160 and a thumb 165 that each receive a finger 145 of the human hand 61 such that each surrounding sidewall 155 has a surrounding sidewall inner surface 170 that has disposed and affixed 175 upon the surrounding sidewall inner surface 170 the electrode 110, wherein each electrode 110 is positioned to contact 180 a fingertip 146 of the human hand 61, see in particular FIGS. 1 to 4.

Alternatively, for the sensory control system, wherein the semi planar garment 80 is constructed of an orthotic arch support shoe insert insole 185 that includes a human foot side first surface 190 and an opposing shoe sole side second surface 195, wherein the human foot side first surface 190 has disposed and affixed 200 upon the human foot side first surface 190 the electrode 110, wherein the electrode 110 is positioned to contact 205 an arch skin surface 77 of the human foot 76, see FIGS. 5 to 8.

A further alternative for the sensory control system 50, wherein the semi planar garment 80 is constructed of a pet grooming type of glove 210 for a human hand 61 that includes a grooming glove 210 surrounding sidewall 215 that is positioned to encompass 220 a human hand 61 palm 62 and a human hand 61 back of the hand 63, the grooming glove surrounding sidewall 215 including an exterior surface 225 that is divided into an inside exterior surface 230 being adjacent 240 to the palm 62 of the hand 61 and an outside exterior surface 235 being adjacent 245 to the back 63 of the hand 61, the inside exterior surface 230 has disposed and affixed 250 upon the inside exterior surface 230 the electrode 110, wherein the electrode 110 is positioned to contact 255 a portion of the human head 78 skin surface 79, see FIGS. 9 to 13.

Accordingly, the present invention of a Sensory Control System has been described with some degree of particularity directed to the embodiments of the present invention. It should be appreciated, though; that the present invention is defined by the following claims construed in light of the prior art so modifications or changes may be made to the exemplary embodiments of the present invention without departing from the inventive concepts contained therein.

1. A sensory control system that is adapted to encompass an outer surface of a particular part of a human body of a user for applying electrical impulses to the outer surface to alter a physical sensation of the particular part of the human body for the user, said sensory control system comprising:
a. a semi planar garment constructed of a flexible material that is adapted to attach to the outer surface of a particular part of the human body, wherein said semi planar garment is removably engageable to the outer surface of a particular part of the human body, said semi planar garment including a planar garment outer surface, a planar garment inner surface, a planar garment first end portion, and an opposing planar garment second end portion, wherein said semi planar garment substantially assumes a shape of a surrounding sidewall;
b. an electrode disposed within said garment, wherein said electrode is affixed to said semi planar garment inner surface, wherein operationally said electrode comes into contact with an outer surface of a particular part of a human body of a user when said semi planar garment is attached to the outer surface of the particular part of the human body; and
c. control circuitry that is in electrical communication with said electrode, wherein said control circuitry is operative to generate the electrical impulses with particular frequency, phase, pulse versus time amounts, voltage, and ampere levels at user safe low electrical power levels that are less than one-hundred milli-amps and no more than sixty volts.

2. A sensory control system according to claim 1 wherein said electrode and said control circuitry are constructed of a transcutaneous electrical nerve stimulation (TENS) system.

3. A sensory control system according to claim 1 wherein said semi planar garment is constructed of a glove for a human hand that includes a plurality of adjacent surrounding sidewalls forming fourchettes and a thumb that each receive a finger of the human hand such that each said surrounding sidewall has a surrounding sidewall inner surface that has disposed and affixed upon said surrounding sidewall inner surface said electrode, wherein each said electrode is positioned to contact a finger tip of the human hand.

4. A sensory control system according to claim 1 wherein said semi planar garment is constructed of an orthotic arch support shoe insert insole that includes a human foot side first surface and an opposing shoe sole side second surface, wherein said human foot side first surface has disposed and affixed upon said human foot side first surface said electrode, wherein said electrode is positioned to contact an arch skin surface of the human foot.

5. A sensory control system according to claim 1 wherein said semi planar garment is constructed of a pet grooming type of glove for a human hand that includes a grooming glove surrounding sidewall that is positioned to encompass a human hand palm and a human hand back of the hand, said grooming glove surrounding sidewall including an exterior surface that is divided into an inside exterior surface being adjacent to the palm of the hand and an outside exterior surface being adjacent to the back of the hand, said inside exterior surface has disposed and affixed upon said inside exterior surface said electrode, wherein said electrode is positioned to contact a portion of the human head skin surface.
